(19) United States
(12) Patent Application Publication
(10) Pub. No.: US 2024/0208890 A1
(43) Pub. Date: Jun. 27, 2024

(54) RECYCLED CONTENT OXO PRODUCTS

(71) Applicant: Eastman Chemical Company, Kingsport, TN (US)

(72) Inventors: Daryl Bitting, Longview, TX (US); David Eugene Slivensky, Tatum, TX (US); Xianchun Wu, Longview, TX (US)

(73) Assignee: Eastman Chemical Company, Kingsport, TN (US)

(21) Appl. No.: 18/567,802

(22) PCT Filed: Jun. 8, 2022
(86) PCT No.: PCT/US2022/032602

§ 371 Date: Dec. 7, 2023

Related U.S. Application Data
(60) Provisional application No. 63/202,453, filed on Jun. 11, 2021.

(51) Int. Cl. C07C 45/50; C10J 3/00

(52) U.S. Cl. CPC C07C 45/505; C10J 3/00; C10J 2300/1846

(57) ABSTRACT

Recycled content oxo products are produced using a process and system that applies physical and/or credit-based recycled content from one or more feed materials to the oxo products produced from the feed materials.

### BACKGROUND

Hydroformylation is an important chemical pathway used to produce aldehydes from olefins. Aldehydes are important chemical intermediates since they can be converted into a wide variety of useful chemicals, such as alcohols, acids, esters, and amides. The products of hydroformylation and subsequent reactions (also called “oxo products”) are useful as or in a wide variety of specialty chemicals, lubricants, coatings, paints, plasticizers, solvents, and the like.

The demand for recycled chemical products continues to grow, but there is no clear path to recycled oxo products through mechanical recycling. Thus, there exists a need for a commercial process to produce recycled content oxo products.

### SUMMARY

In one aspect, the present technology concerns a process for producing an oxo product having recycled content, the process comprising: hydroformylating a C2 to C24 olefin with syngas to provide an oxo product, wherein the oxo product comprises recycled content obtained directly or indirectly from waste plastic subjected to carbon reforming.

In one aspect, the present technology concerns a process for producing an oxo product having recycled content, the process comprising: (a) carbon reforming a hydrocarbon containing feed to thereby produce a first syngas; (b) hydroformylating a C2 to C24 olefin with at least a portion of the first syngas to thereby produce a C3 to C25 aldehyde; and (c) subjecting at least a portion of the C3 to C25 aldehyde to one or more additional reactions with an additional reactant to provide an oxo product, wherein the oxo product comprises recycled content from one or more of the following sources: (i) waste plastic, (ii) a recycled content syngas (r-syngas), and (iii) a recycled content additional reactant.

In one aspect, the present technology concerns a process for producing an oxo product or a derivative thereof having recycled content, the process comprising: (a) carbon reforming a hydrocarbon containing feed to thereby produce a first syngas; (b) hydroformylating a C2 to C24 olefin with at least a portion of the first syngas to thereby produce an oxo product; and (c) subjecting at least a portion of the oxo product to at least one additional reaction with an additional reactant to thereby produce an oxo product derivative, wherein the oxo product derivative comprises recycled content from one or more of the following sources: (i) waste plastic, (ii) a recycled content syngas (r-syngas), and (iii) a recycled content additional reactant.

In one aspect, the present technology concerns a system or package comprising: an oxo product and an identifier associated with the oxo product, wherein the identifier is a representation that the oxo product has recycled content or is made from a source having recycled content.

In one aspect, the present technology concerns the use of a recycled content syngas to produce a recycled content oxo product.

In one aspect, the present technology concerns a recycled content end product comprising a recycled content oxo product and at least one other material.

### BRIEF DESCRIPTION OF THE DRAWINGS

FIG. 1a is a block flow diagram illustrating the main steps of a process and facility for making a recycled content oxo product (r-oxo product);

FIG. 1b is a block flow diagram illustrating the main steps of a process and facility for making an r-oxo product, particularly showing an embodiment where at least a portion of the olefin comes from the pyrolysis of waste plastic;

FIG. 1c is a block flow diagram illustrating the main steps of a process and facility for making an r-oxo product and a recycled content oxo product derivative (r-oxo product derivative); and

FIG. 2 is a block flow diagram illustrating the main steps of a process and facility for making an r-oxo product, where the r-oxo product has credit-based recycled content from one or more source materials.

### DETAILED DESCRIPTION

We have discovered new methods and systems for producing oxo products having recycled content. As used herein, the term “oxo product” refers to the product of a hydroformylation reaction and chemical derivatives thereof. Some examples of oxo products include, for example, aldehydes, alcohols, carboxylic acids, ketones, esters, amides, ethers, amines, olefins (alkenes), and paraffins (alkanes). More specifically, we have discovered a process and system for producing oxo products where recycled content from waste materials, such as waste plastic, is applied to oxo products in a manner that promotes the recycling of waste plastic and provides oxo products with substantial amounts of recycled content.

In general, oxo products are formed by hydroformylating a C2 to C24 olefin with syngas to form an aldehyde. The aldehyde can be withdrawn as an oxo product stream or further reacted in one or more additional reactions downstream of hydroformylation. Examples of these additional reactions can include, but are not limited to, condensation (such as aldol condensation), hydrogenation, dehydration, addition (transesterification), oxidation, esterification, amination, carbonylation, Tischenko reaction and combinations thereof. The resulting oxo product from the downstream reactions can comprise a carboxylic acid, a ketone, an ester, an amide, an ether, an amine, an olefin (alkene), or a paraffin (alkane). In some embodiments, the aldehyde or other oxo product can be subjected to two or more, three or more, or even four or more additional reactions after hydroformylation to provide a final oxo product.

In some embodiments, at least a portion of the aldehyde can be subjected to hydrogenation to form an alcohol. The alcohol can be withdrawn as a final product and/or it may be subjected to additional reactions to form further oxo products. The resulting oxo product from these additional downstream reactions can comprise a carboxylic acid, a ketone, an ester, an amide, an ether, an amine, an olefin (alkene), or a paraffin (alkane). In some embodiments, the alcohol or other oxo product can be subjected to two or more, three or more, or even four or more additional reactions after hydroformylation or hydrogenation to provide a final oxo product.

The oxo products formed at the facility can include recycled content from one or more source materials including, for example, waste plastic, recycled content syngas (r-syngas), recycled content hydrogen (r-H2), and one or more recycled content reactants (r-reactants) reacted with the oxo product in one or more additional reactions. The recycled content in the oxo products can be physical and may directly originate from at least one of these streams, and/or the recycled content may be credit-based (e.g., indirectly obtained from one or more of these streams) and applied to a target stream in the process of making the oxo product from one or more of these source streams.

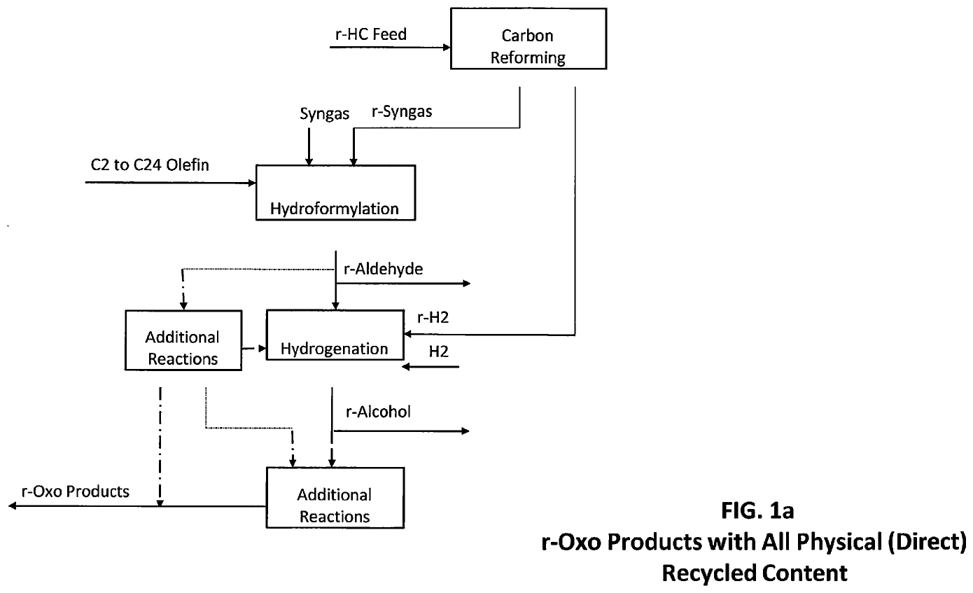

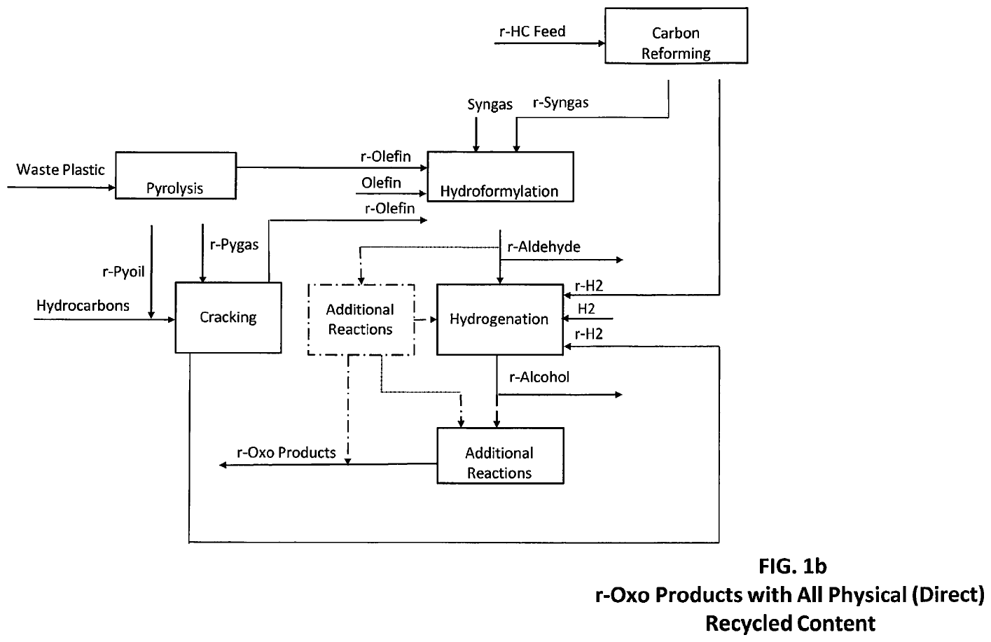

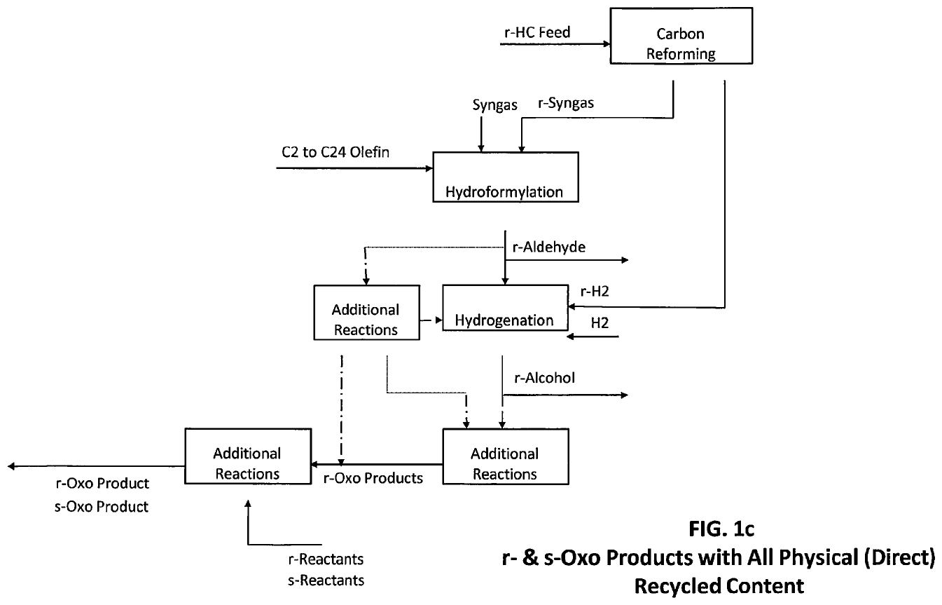

Turning now to FIGS. 1a-c, several embodiments of a process and facility for forming an oxo product with physical (direct) recycled content are provided. The recycled content in the oxo product can originate from carbon reforming of a recycled content hydrocarbon-containing feed (FIGS. 1a-c), and/or from the pyrolysis of (and/or cracking of pyrolyzed) waste plastic (FIG. 1b). Alternatively, or in addition, the recycled content in the oxo product can originate from a recycled content reactant (r-reactant) utilized in one or more additional reactions performed downstream of hydroformylation (FIG. 1c). The resulting oxo product from one or more of these embodiments can have a total recycled content of at least 5, at least 10, at least 25, at least 35, or at least 45 percent and/or less than 99, less than 95, less than 90, or less than 85 percent. Or the total recycled content can be at least 50, at least 60, at least 70, at least 75, at least 80, at least 85, at least 90, or at least 95 percent.

Turning now to FIGS. 1a-c, a stream of recycled content hydrocarbon feed (r-HC feed) can be subjected to carbon reforming to produce a recycled content syngas (r-syngas) and a recycled content hydrogen (r-H2) each having physical recycled content. In some embodiments, the feed to carbon reforming can comprise both a recycled content feed component (e.g., waste plastic) and a non-recycled content feed component (e.g., coal, a liquid hydrocarbon, and/or a gaseous hydrocarbon). In one embodiment, the carbon reforming is partial oxidation gasification that is fed with coal and waste plastic. In another embodiment, the carbon reforming is plasma gasification of a predominately waste plastic feed. In yet another embodiment, the carbon reforming is partial oxidation gasification fed with a non-recycled content liquid or gaseous hydrocarbon and a recycled content pyrolysis oil produced from the pyrolysis of waste plastic. In some embodiments, the carbon reforming can include catalytic reforming, while in other embodiments, the carbon reforming can include steam reforming. In another embodiment, the oxo product comprises recycled content obtained directly or indirectly from waste plastic subjected to carbon reforming. In another embodiment, the carbon reforming comprises partial oxidation gasification, or catalytic reforming, or steam reforming, or plasma gasification

“As used herein, the term “partial oxidation” refers to high temperature conversion of a carbon-containing feed into syngas (carbon monoxide, hydrogen, and carbon dioxide), where the conversion is carried out with an amount of oxygen that is less than the stoichiometric amount of oxygen needed for complete oxidation of carbon to CO2. The feed to POX gasification can include solids, liquids, and/or gases.

In some embodiments, the carbon reforming may include a gasifier that can comprise a gas-fed gasifier, a liquid-fed gasifier, a solid-fed gasifier, or a combination thereof. More particularly, the carbon reforming can include liquid-fed partial oxidation gasification. As used herein, “liquid-fed partial oxidation gasification” refers to a partial oxidation gasification process where the feed to the process comprises predominately components that are liquid at 25° C. and 1 atm. Additionally, the carbon reforming may comprise gas-fed partial oxidation gasification. As used herein, “gas-fed partial oxidation gasification” refers to a partial oxidation gasification process where the feed to the process comprises predominately components that are gaseous at 25° C. and 1 atm.

In some embodiments, the carbon reforming includes partial oxidation gasification. During partial oxidation gasification, the gasifier is operated in an oxygen-lean environment, relative to the amount needed to completely oxidize 100 percent of the carbon and hydrogen bonds. For example, the total oxygen requirements for the gasifier may be at least 5, 10, 15, or 20 percent in excess of the amount theoretically required to convert the carbon content of the gasification feedstock to carbon monoxide. In general, satisfactory operation may be obtained with a total oxygen supply of 10 to 80 percent in excess of the theoretical requirements. For example, examples of suitable amounts of oxygen per pound of carbon may be in the range of 0.4 to 3.0, 0.6 to 2.5, 0.9 to 2.5, or 1.2 to 2.5 pounds free oxygen per pound of carbon.

When partial oxidation gasification is used for the carbon reforming step, the type of gasification technology employed can be a partial oxidation entrained flow gasifier that generates syngas. This technology is distinct from fixed bed (alternatively called moving bed) gasifiers and from fluidized bed gasifiers. In fixed bed (or moving bed gasifiers), the feedstock stream moves in a countercurrent flow with the oxidant gas, and the oxidant gas typically employed is air. The feedstock stream falls into the gasification chamber, accumulates, and forms a bed of feedstock. Air (or alternatively oxygen) flows from the bottom of the gasifier up through the bed of feedstock material continuously while fresh feedstock continuously falls down from the top by gravity to refresh the bed as it is being combusted. The combustion temperatures are typically below the fusion temperature of the ash and are non-slagging.

Whether the fixed bed operated in countercurrent flow or in some instances in co-current flow, the fixed bed reaction process generates high amount of tars, oils, and methane produced by pyrolysis of the feedstock in the bed, thereby both contaminating the syngas produced and the gasifier. The contaminated syngas requires significant effort and cost to remove tarry residues that would condense once the syngas is cooled, and because of this, such syngas streams are generally not used to make chemicals and are instead used in direct heating applications.

In a fluidized bed, the feedstock material in the gasification zone is fluidized by action of the oxidant flowing through the bed at a high enough velocity to fluidize the particles in the bed. In a fluidized bed, the homogeneous reaction temperatures and low reaction temperatures in the gasification zone also promotes the production of high amounts of unreacted feedstock material and low carbon conversion, and operating temperatures in the fluidized bed are typically between 800-1000° C. Further, in a fluidized bed it is important to operate below slagging conditions to maintain the fluidization of the feedstock particles which would otherwise stick to the slag and agglomerate. By employing an entrained flow gasification, these deficiencies present with fixed (or moving bed) and fluidized bed gasifiers that are typically used to process waste materials is overcome. An exemplary gasifier that may be used in depicted in U.S. Pat. No. 3,544,291, the entire disclosure of which is incorporated herein by reference in its entirety.

In some embodiments, the gasifier can be non-catalytic, meaning that the gasifier does not contain a catalyst bed and the gasification process is non-catalytic, meaning that a catalyst is not introduced into the gasification zone as a discrete unbound catalyst. Furthermore, in one embodiment or in combination with any of the mentioned embodiments, the gasification process is also a slagging gasification process; that is, operated under slagging conditions (well above the fusion temperature of ash) such that a molten slag is formed in the gasification zone and runs along and down the refractory walls.

The gasification zone, and optionally all reaction zones in the gasifier, are operated at a temperature of at least 1000° C., 1100° C., 1200° C., 1250° C., or 300° C. and/or not more than 2500° C., 2000° C., 1800° C., or 1600° C. The reaction temperature can be autogenous. Advantageously, the gasifier operating in steady state mode is at an autogenous temperature and does not require application of external energy sources to heat the gasification zone.

In some embodiments, the gasifier is a predominately gas fed gasifier. In some embodiments, the gasifier is a non-slagging gasifier or operated under conditions not to form a slag.

In some embodiments, the gasifier is not under negative pressure during operations, but rather is under positive pressure during operation. For example, the gasifier can be operated at a pressure within the gasification zone (or combustion chamber) of at least 200 psig (1.38 MPa), 300 psig (2.06 MPa), 350 psig (2.41 MPa), 400 psig (2.76 MPa), 420 psig (2.89 MPa), 450 psig (3.10 MPa), 475 psig (3.27 MPa), 500 psig (3.44 MPa), 550 psig (3.79 MPa), 600 psig (4.13 MPa), 650 psig (4.48 MPa), 700 psig (4.82 MPa), 750 psig (5.17 MPa), 800 psig (5.51 MPa), 900 psig (6.2 MPa), 1000 psig (6.89 MPa), 1100 psig (7.58 MPa), or 1200 psig (8.2 MPa).

Generally, the average residence time of gases in the gasifier reactor can be very short to increase throughput. The average residence time of the gases in the gasifier can be not more than 30, 25, 20, 15, 10, or 7 seconds.

In some embodiments, when the carbon reforming includes gasification, the gasifier can include at least the following properties: (i) single stage; (ii) slagging; (iii) downflow; (iv) entrained flow; (v) high pressure; (vi) high temperature; (vii) slurry fed; (viii) coal or PET fed; and/or (ix) quenching gasifier.

In some embodiments, all or a portion of the r-syngas from carbon reforming may be used in hydroformylation. The olefin subjected to hydroformylation can include at least 50, at least 75, at least 90, or at least 95 weight percent of C2 to C24 olefin, C2 to C20 olefin, or C3 to C16 olefin, or C2 olefin, C3 olefin, C4 olefin, C5 olefin, or C6 olefin. As used herein, the term “Cx” or “Cx hydrocarbon,” refers to a hydrocarbon compound including “x” total carbons per molecule, and encompasses all olefins, paraffins, aromatics, heterocyclic, and isomers having that number of carbon atoms. For example, each of normal, iso, and tert-butane and butene and butadiene molecules would fall under the general description “C4.”

In some embodiments (shown, for example, in FIGS. 1a and 1c), the olefin may have non-recycled content. In other embodiments, the olefin may comprise recycled content, which can originate from the pyrolysis of waste plastic and/or from the cracking of recycled content pyrolysis oil (r-pyoil) and/or separation of recycled content pyrolysis gas (r-pygas) formed during the pyrolysis of waste plastic. For example, as shown in FIG. 1b, in some embodiments, waste plastic can be pyrolyzed to form recycled content pyrolysis gas (r-pygas) and recycled content pyrolysis oil (r-pyoil). All or a part of the r-pygas and/or r-pyoil can be introduced into a cracking facility, where it can be used to produce a recycled content olefin. Alternatively, or in addition, recycled content olefin may also be separated out in the pyrolysis facility. The feed to the cracking facility can include only r-pyoil and/or r-pygas, or it can also include non-recycled content hydrocarbon, such as naphtha (e.g., C5 to C22) or lighter hydrocarbon components (e.g., C2 to C5). The total amount of olefin fed to the hydroformylation can include at least 25, at least 40, at least 50, at least 75, at least 90, or 100 percent recycled content. In one embodiment, the olefin is a recycled content olefin (r-olefin) formed by pyrolysis of waste plastic, methanol, or cracking of hydrocarbons.

In some embodiments, both the olefin and the syngas used for hydroformylation can have recycled content, while in other embodiments, one or both of the olefin and syngas may have non-recycled content. In some embodiments, the olefin and/or syngas can include both recycled and non-recycled content. The total amount of syngas fed to hydroformylation can include at least 25, at least 40, at least 50, at least 75, at least 90, or 100 percent recycled content.

Hydroformylation of a C2 to C24 olefin can provide a C3 to C25 aldehyde. In some embodiments, the olefin can comprise C2 olefin and the aldehyde a C3 aldehyde, while in other embodiments, the olefin can comprise C3 olefin and the aldehyde can comprise a C4 olefin (normal and/or iso-C4 olefin). The aldehyde can comprise at least 50, at least 75, at least 90, or at least 95 weight percent of a C3, iC4, or n-C4 olefin. The aldehyde can be a recycled content aldehyde (r-aldehyde) and can include at least 25, at least 50, at least 75, or at least 90 percent recycled content from at least one source material such as waste plastic, r-hydrocarbon feed, r-syngas, and/or r-olefin. In some cases, most or all of the recycled content applied to the r-aldehyde originates from the r-syngas fed to hydroformylation.

As shown in FIGS. 1a-c, at least a portion or all of the aldehyde formed during hydroformylation can be withdrawn as a product stream, and/or it can be further reacted in one or more additional downstream reactions to form another oxo product. In one embodiment, the additional reaction can include hydrogenation to convert the aldehyde to an alcohol. All or a portion of the hydrogen used for hydrogenating the aldehyde can include recycled content hydrogen (r-H2). When used, the r-H2 can originate from carbon reforming of r-HC feed (as shown in FIG. 1a) and/or from cracking pyrolyzed waste plastic (as shown in FIG. 1b). In some embodiments, at least portion or all of the hydrogen can be non-recycled content hydrogen. The total amount of hydrogen fed to hydrogenate the aldehyde can include at least 25, at least 40, at least 50, at least 75, at least 90, or 100 percent recycled content.

Alternatively (or in addition) to hydrogenation, the aldehyde (or alcohol) can be subjected to one or more additional reactions. Such reactions can include, but are not limited to, condensation (such as aldol condensation), hydrogenation, dehydration, oxidation, esterification, amination, and combinations thereof. The oxo product from these additional reactions can comprise a hydrocarbon such as an alcohol, an aldehyde, a carboxylic acid, a ketone, an ester, an amide, an ether, an amine, an olefin (alkene), or a paraffin (alkane). The oxo product can comprise a C3 to C50 hydrocarbon, a C3 to C35 hydrocarbon, a C3 to C30 hydrocarbon, or a C4 to C18 hydrocarbon.

In one or more embodiments, the additional reaction can be a polymerization reaction and the oxo product can comprise a polymer. For example, in some embodiments, the oxo product can comprise a polyester.

Turning now to FIG. 1c, an embodiment in which the oxo product (including a recycled content oxo product) is further reacted with at least one additional reactant to form another oxo product is shown. In some embodiments, the additional reactant can comprise recycled content (r-reactant), while in others, the additional reactant can comprise sustainable content (s-reactant). As used herein, the term “sustainable content” refers to content originating from a natural source. The oxo product produced by such an additional reaction can have recycled content, sustainable content, or both recycle and sustainable content.

Examples of suitable types of additional reactants can include, but are not limited to, hydrogen, formaldehyde, acetaldehyde, ethylene oxide, acetic acid, acetic anhydride, acetone, and benzoic acid. One or more of these reactants, when used, can include recycled content such that the r-reactant can comprise recycled content hydrogen (r-H2), recycled content formaldehyde (r-formaldehyde), recycled content acetaldehyde (r-acetaldehyde), recycled content ethylene oxide (r-EO), recycled content acetic acid (r-acetic acid), recycled content acetic anhydride (r-acetic anhydride), recycled content acetone (r-acetone), and recycled content benzoic acid (r-benzoic acid). The recycled content in each of these reactants can originate from one or more processes for chemically recycling waste plastic including, but not limited to, pyrolysis, cracking, solvolysis, carbon reforming, and combinations thereof. Further reactions can be performed on the products from one or more of these chemical recycling products to produce the recycled content reactants.

For example, in one embodiment, the additional reactant can comprise dimethyl terephthalate (DMT). All or a portion of the DMT can be recycled content DMT (r-DMT) and may originate from, for example, the solvolysis of waste plastic including polyethylene terephthalate (PET). In another embodiment, the additional reactant can comprise ethylene glycol (EG), all or a portion of which can be recycled content ethylene glycol. In some embodiments, at least a portion of the ethylene glycol can originate from the solvolysis of waste plastic including polyethylene terephthalate (PET). In another embodiment, the additional reactant can comprise recycled content dimethyl terephthalate (r-DMT) or recycled content ethylene glycol (r-EG). The additional reactant can have a recycled content of at least 50, at least 75, at least 90, or at least 95 percent, or it can have a recycled content of 100 percent.

In one or more embodiments, the additional reactant can be a sustainable reactant (s-reactant) having at least 50, at least 75, at least 90, or at least 95, or 100 percent sustainable content. Examples of s-reactants can include, but are not limited to, cellulose and naturally occurring acids and alcohols (e.g., C10 to C22 alcohols and carboxylic acids).

In one or more embodiments, the olefin can comprise a predominantly C2 olefin, which when hydroformylated, can provide a C3 aldehyde. When subjected to one or more additional reactions, the additional oxo product can be one or more of the following: n-propanol, n-propyl acetate, glycol ether, n-propyl propionate, cellulose acetate propionate, propionic acid, and propionic anhydride.

In one or more embodiments, the olefin can comprise a predominantly C3 olefin and, when hydroformylated, can produce a C4 aldehyde. The aldehyde can comprise at least 50, at least 75, at least 90, or at least 95 percent of an n-C4 or an iC4 aldehyde. When subjected to one or more additional reactions, the additional oxo product can be one or more of the following compounds: 2-ethylhexanol, 2-ethylhexaldehyde, 2-ethylhexylacid, tri(ethylene glycol)bis(2-ethanolhexanoate), and 2-ethylhexanol acetate.

The additional oxo product can include at least one of dipropylene glycol dibenzoate, bis(2-ethylhexyl) maleate, bis(2-ethylhexyl) terephthalate, dioctyl phthalate, bis(2-ethylhexyl) adipate, oxydiethylene dibenzoate, tris(2-ethylhexyl)trimellitate, and dibutylterephthalate.

The additional oxo product can includes one or more of the following compounds: methyl n-amyl ketone (MAK), n-butyric acid, normal butanitrile, butyric anhydride, and cellulose acetate butyrate.

The additional oxo product can include one or more of the following compounds: n-butanol, n-butyl propionate, n-butyl acetate, n-butyraldehyde, ethylene glycol monobutyl ether, diethylene glycol monobutyl ether, ethylene glycol monopropyl ether, ethylene glycol dipropyl ether, ethylene glycol 2-ethylhexyl ether, glycol ether esters (ethylene glycol monobutyl ether ester, diethylene glycol monobutyl ether ester, and diethylene glycol monoethyl acetate).

The additional oxo product can include one or more of the following compounds: methyl isoamyl ketone, neopentyl glycol, 2,2,4-trimethyl-1,3-pentanediol monoisobutyrate, 2,2,4-trimethyl-1,3-pentanediol diisobutyrate, 2,2,4-trimethyl-1,3-pentanediol, isobutyl isobutyrate, isobutyl alcohol, isobutyl acetate, 2,2,4,4-tetramethyl-1,3-cyclobutanediol, isobutyric anhydride isobutyronitrile, and methyl isopropyl ketone.

The additional oxo product can include one or more of the following compounds: n-propanol, n-propyl acetate, glycol ether, n-propyl propionate, cellulose acetate propionate, propionic acid, and propionic anhydride, 2-ethylhexanol, 2-ethylhexaldehyde, 2-ethylhexylacid, tri(ethylene glycol)bis(2-ethanolhexanoate), 2-ethylhexanol acetate, dipropylene glycol dibenzoate, bis(2-ethylhexyl) maleate, bis(2-ethylhexyl) terephthalate, dioctyl phthalate, bis(2-ethylhexyl) adipate, oxydiethylene dibenzoate, tris(2-ethylhexyl)trimellitate, dibutylterephthalate, methyl n-amyl ketone (MAK), n-butyric acid, normal butranitrile, butyric anhydride, cellulose acetate butyrate, n-butanol, n-butyl propionate, n-butyl acetate, n-butyraldehyde, ethylene glycol monobutyl ether, diethylene glycol monobutyl ether, ethylene glycol monopropyl ether, ethylene glycol dipropyl ether, ethylene glycol 2-ethylhexyl ether, glycol ether esters (ethylene glycol monobutyl ether ester, diethylene glycol monobutyl ether ester, and diethylene glycol monoethyl acetate), methyl isoamyl ketone, neopentyl glycol, 2,2,4-trimethyl-1,3-pentanediol monoisobutyrate, 2,2,4-trimethyl-1,3-pentanediol diisobutyrate, 2,2,4-trimethyl-1,3-pentanediol, isobutyl isobutyrate, isobutyl alcohol, isobutyl acetate, 2,2,4,4-tetramethyl-1,3-cyclobutanediol, isobutyric anhydride, isobutyronitrile, and methyl isopropyl ketone, and copolyester.

The oxo product can have a recycled content of at least 5, at least 10, at least 15, at least 20, at least 25, at least 30, at least 35, at least 40, at least 45, at least 50, at least 55, or at least 65 percent and/or 100 percent, or less than 99, less than 95, less than 90, less than 85, less than 80, less than 75, or less than 70 percent. In some embodiments, the oxo product can have a recycled content of at least 70, at least 75, at least 80, at least 85, at least 90, at least 95, or 100 percent.

The amount of physical recycled content in the r-oxo product can determined by tracing the amount of recycled material along a chemical pathway starting with waste plastic and ending with the oxo product. The chemical pathway includes all chemical reactions and other processing steps (e.g., separations) between the starting material (e.g., waste plastic) and the oxo product. In FIG. 1a, the chemical pathway can include one or more of carbon reforming, hydroformylation, and one or more additional reactions as described herein, depending on the exact oxo product being produced.

In one or more embodiments, a conversion factor can be associated with each step along the chemical pathway. The conversion factors account for the amount of the recycled content diverted or lost at each step along the chemical pathway. For example, the conversion factors can account for the conversion, yield, and/or selectivity of the chemical reactions along the chemical pathway.

The amount of recycled content applied to the r-oxo product can be determined using one of variety of methods for quantifying, tracking, and allocating recycled content among various materials in various processes. One suitable method, known as “mass balance,” quantifies, tracks, and allocates recycled content based on the mass of the recycled content in the process. In certain embodiments, the method of quantifying, tracking, and allocating recycled content is overseen by a certification entity that confirms the accuracy of the method and provides certification for the application of recycled content to the r-oxo product.

The r-oxo product can include recycled content from the r-syngas. The chemical pathway can include, for example, carbon reforming, hydroformylation, and any further reactions (e.g., hydrogenation). The recycled content from the r-syngas can be physical recycled content, credit-based recycled content, or a combination of physical and credit-based recycled content. In one embodiment, the r-oxo product (or oxo product) comprises recycled content from the r-syngas, or physical recycled content from the r-syngas, or credit-based recycled content from the r-syngas or recycled content from the r-H2, or physical recycled content from the r-H2 or credit-based recycled content from the r-H2, or recycled content from the r-reactant or physical recycled content from the r-reactant, or a combination.

The r-oxo product can include recycled content from the r-hydrogen, particularly when the aldehyde from hydroformylation is hydrogenated to form an alcohol. The chemical pathway can include, for example, carbon reforming and hydrogenation. The recycled content from the r-H2 can be physical recycled content, credit-based recycled content, or a combination of physical and credit-based recycled content.

The r-oxo product can comprise recycled content from the r-additional reactant (and/or sustainable content from the s-reactant). The chemical pathway can include, for example, hydroformylation and at least one additional reaction as described herein (depending on the specific oxo product being produced). The recycled content from the additional reactant (r-reactant) and/or the sustainable content from the s-reactant can be physical recycled content, credit-based recycled content, or a combination of physical and credit-based recycled content.

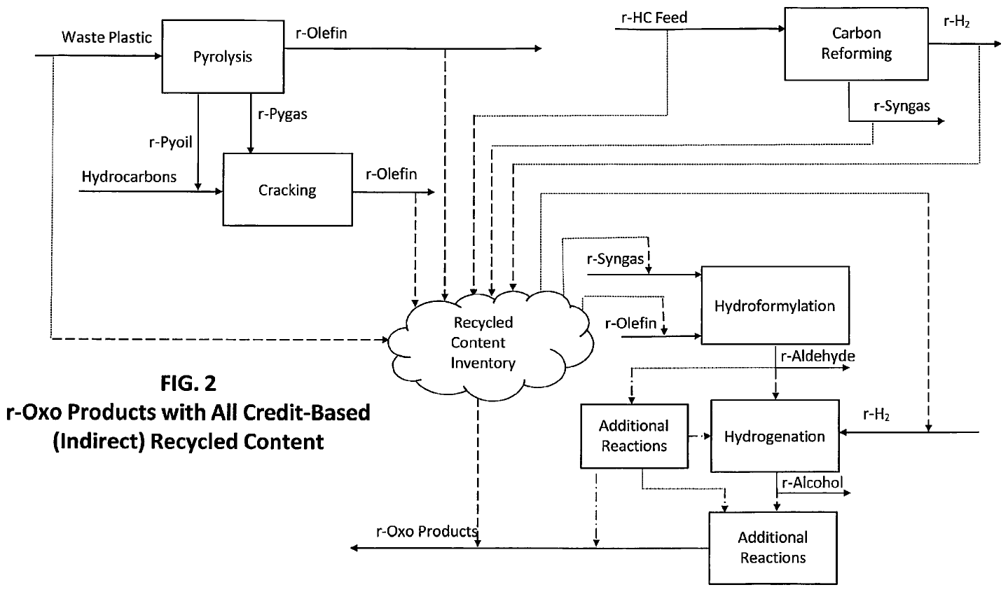

Turning now to FIG. 2, an embodiment where the r-oxo product has no physical recycled content, but has credit-based recycled content, is provided. In the process and system depicted in FIG. 2, the r-syngas (and, in some embodiments, the r-olefin) are not fed directly into the hydroformylation. Additionally, r-H2 is not directly used in the hydrogenation, as shown in the embodiment depicted in FIG. 2.

Instead, recycled content credits from the recycled content streams shown in FIG. 2 (e.g., the r-syngas and the r-H2 and r-olefin where used, as well as the waste plastic and/or r-HC feed) can be attributed to one or more streams within the production facility. For example, the recycled content credits from one or more of the above streams can be attributed to the syngas (and/or olefin) fed to the hydroformylation and/or to the hydrogen fed to the hydrogenation. As such, the r-syngas, the r-H2, and the r-olefin, when used, each act as a “source material” of recycled content credits, and the syngas fed to the hydroformylation and the hydrogen fed to the hydrogenation and/or olefin fed to hydroformylation can each act as a “target material” to which the recycled content credits are attributed.

In one or more embodiments, the source material has physical recycled content and the target material has less than 100 percent physical recycled content. For example, the source material can have at least 10, at least 25, at least 50, at least 75, at least 90, at least 99, or 100 percent physical recycled content and/or the target material can have less than 100, less than 99, less than 90, less than 75, less than 50, less than 25, less than 10, or less than 1 percent physical recycled content.

The ability to attribute recycled content credits from a source material to a target material removes the co-location requirement for the facility making the source material (with physical recycled content) and the facility making the oxo product. This allows a chemical recycling facility/site in one location to process waste material into one or more recycled content source materials and then apply recycled content credits from those source materials to one or more target materials being processed in existing commercial facilities located remotely from the chemical recycling facility/site. Further, the use of recycled content credits allows different entities to produce the source material and the r-oxo product. This allows efficient use of existing commercial assets to produce r-oxo products. In one or more embodiments, the source material is made at a facility/site that is at least 0.1, at least 0.5, at least 1, at least 5, at least 10, at least 50, at least 100, at least 500, or at least 1000 miles from the facility/site where the target material is used to make the oxo product.

The attributing of recycled content credits from the source material (e.g., the r-syngas produced by carbon reforming of recycled content hydrocarbon feed) to the target material (e.g., the syngas fed to hydroformylation) can be accomplished by transferring recycled content credits directly from the source material to the target material. Alternatively, as shown in FIG. 2, recycled content credits can be applied from any of the waste plastic, the recycled content hydrocarbon feed to the carbon reforming step (r-HC Feed), the r-syngas, the r-H2, and/or the r-olefin to the oxo product via a recycled content inventory. The recycled content inventory can be a digital inventory or database used to record and track recycled content for various materials at various sites over various time periods.

When a recycled content inventory is used, recycled content credits from the source material having physical recycled content (e.g., the waste plastic, the r-HC Feed, the r-olefin, the r-syngas, and/or the r-H2 in FIG. 2) are booked into the recycled content inventory. The recycled content inventory can also contain recycled content credits from other sources and from other time periods.

In one embodiment, recycled content credits in the recycled content inventory can only be assigned to target materials having the same or similar composition as the source materials. For example, as shown in FIG. 2, recycled content credits booked into the recycled content inventory from the r-syngas from carbon reforming can be assigned to the syngas fed into hydroformylation because the two syngas streams have the same or similar compositions. However, recycled content credits from r-syngas could not be assigned to the hydrogen fed to the hydrogenation step or to an additional reactant fed to a downstream reaction because the source and target materials would not be the same or similar.

In some embodiments, all or a portion of the recycled content credit can be applied to one or more target materials (e.g., syngas) upon receipt of one or more waste plastic containing materials at the facility. That is, the waste plastic (or recycled content hydrocarbon feed) need not be processed before applying the credit-based recycled content to the target material. Instead, receipt of the waste plastic (or waste-plastic containing material) at the facility can permit application of recycled content credit to one or more target materials. In most cases, however, such waste plastic will then be processed at the facility within 30, 60, or 90 days to produce one or more of the target materials.

Once recycled content credits have been attributed to the target material (e.g., the syngas or the hydrogen), the amount of the credit-based recycled content allocated to the oxo product is calculated by tracing the recycled content along the chemical pathway from the target material to the oxo product. The chemical pathway includes all chemical reactions and other processing steps (e.g., separations) between the target material and the oxo product, and a conversion factor can be associated with each step along the chemical pathway of the credit-based recycled content. The conversion factors account for the amount of the recycled content diverted or lost at each step along the chemical pathway. For example, the conversion factors can account for the conversion, yield, and/or selectivity of the chemical reactions along the chemical pathway.

As with the physical recycled content, the amount of credit-based recycled content applied to the r-oxo product can be determined using one of variety of methods, such as mass balance, for quantifying, tracking, and allocating recycled content among various materials in various processes. In certain embodiments the method of quantifying, tracking, and allocating recycled content is overseen by a certification entity that confirms the accuracy of the method and provides certification for the application of recycled content to the r-oxo product. In one embodiment, the oxo product comprises physical recycled content from one or more of the source materials and/or credit-based recycled content from one or more of the source materials.

The r-oxo product can have 25 to 90, 40 to 80, or 55 to 65 percent credit-based recycled content and less than 50, less than 25, less than 10, less than 5, or less than 1 percent physical recycled content. In certain embodiments, the r-oxo product can have 10 to 80, 20 to 75, or 25 to 70 percent credit-based recycled content from one or more of the r-syngas, the r-H2, and the r-olefin, individually.

In one or more embodiments, the recycled content of the r-oxo product can include both physical (direct) recycled content and credit-based (indirect) recycled content. For example, the r-oxo product can have at least 10, at least 20, at least 30, at least 40, or at least 50 percent physical recycled content and at least 10, at least 20, at least 30, at least 40, or at least 50 percent credit-based recycled content. As used herein, the term “total recycled content” refers to the cumulative amount of physical recycled content and credit-based recycled content from all sources.

In some embodiments, both physical recycled content and credit-based recycled content can be attributed to the r-oxo product. Any combination of physical recycled content shown in FIGS. 1a-c and credit-based recycled content shown in FIG. 2 can be used to form and/or can be attributed to one or more oxo products to thereby produce r-oxo products.

For example, physical recycled content can be supplied by at least 1, at least 2, at least 3, at least 4, at least 5, or all of the sources shown in FIGS. 1a-c, including the waste plastic, the r-HC feed, the r-olefin, the r-H2, and/or the r-syngas, while the credit-based recycled content can be supplied by one or more of the other sources shown in FIG. 2. In some embodiments, the r-oxo product can include 10 to 60, 20 to 50, or 25 to 40 percent physical recycled content and 10 to 60, 20 to 50, or 25 to 40 percent credit-based recycled content. Alternatively, the r-oxo product can include less than 15, less than 10, or less than 5 percent physical recycled content (or credit-based recycled content) and at least 85, at least 90, or at least 95 percent credit based recycled content (or physical recycled content).

For example, in some embodiments, physical recycled content can be provided by r-syngas fed to hydroformylation, while credit-based recycled content can be provided by r-H2. In other embodiments, physical recycled content can be provided by r-H2, while credit-based recycled content can be provided by r-syngas. Alternatively, the recycled content applied to the r-oxo product can originate from the r-reactant added in downstream reactions.

Turning again to the embodiment shown in FIG. 1, the carbon reforming facility and/or one or more of the additional reaction facilities can be co-located with the hydroformylation facility. When a pyrolysis/cracking facility are present, these may also be co-located. In the embodiment shown in FIG. 2, the carbon reforming facility and/or one or more of the additional reaction facilities can be remotely located from the hydroformylation facility. In some embodiments, the portions of the carbon reforming and/or additional reaction facilities (and, when applicable, the pyrolysis/cracking facilities) providing physical recycled content to the hydroformylation facility may be co-located, while the portions of the facilities providing credit-based recycled content to the hydroformylation facility may be remotely located. When remotely located, the two facilities can be at least 0.5, 1, 5, 10, 100, 500, 1000, or 10,000 miles from the other. When co-located, the two facilities can be within 5, 1, 0.5, or 0.25 miles of one another. As discussed previously, when remotely located, two or more of the facilities may be owned and/or operated by the same or by different commercial entities.

The resulting recycled content oxo product can be used in a variety of end use applications. In some cases, the r-oxo product can be refined/purified and may be employed as itself. For example, the oxo product may be used as a solvent, a surfactant, a plasticizer, a lubricant, a coalescent, a dye, a fragrance, or a polymerizing agent or additive.

In other cases, the r-oxo product can be incorporated with one or more other materials to produce a recycled content end use product. Such end use products can be utilized in medical, consumer, commercial, or industrial end uses. Examples of such products can include, but are not limited to, personal care product, a fragrance, detergent, cosmetic, a solvent, a coating, a paint, a fiber, a packaging material, a bottle, a container, a sheet, construction materials, a medical product (e.g., eyewear), an electronic product (e.g., displays), a food additive (e.g., preservative), and an automobile component (e.g., plastics, carpets, windshield interlayers, fluids).

### Definitions

It should be understood that the following is not intended to be an exclusive list of defined terms. Other definitions may be provided in the foregoing description, such as, for example, when accompanying the use of a defined term in context.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the phrase “at least a portion” includes at least a portion and up to and including the entire amount or time period.

As used herein, the term “chemical pathway” refers to the chemical processing step or steps (e.g., chemical reactions, physical separations, etc.) between an input material and a product material, where the input material is used to make the product material.

As used herein, the term “chemical recycling” refers to a waste plastic recycling process that includes a step of chemically converting waste plastic polymers into lower molecular weight polymers, oligomers, monomers, and/or non-polymeric molecules (e.g., hydrogen, carbon monoxide, methane, ethane, propane, ethylene, and CO) that are useful by themselves and/or are useful as feedstocks to another chemical production process(es).

As used herein, the term “co-located” refers to the characteristic of at least two objects being situated on a common physical site, and/or within 5, 1, 0.5, or 0.25 miles of each other.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the terms “credit-based recycled content,” “non-physical recycled content,” and “indirect recycled content” all refer to matter that is not physically traceable back to a waste material, but to which a recycled content credit has been attributed.

As used herein, the term “directly derived” refers to having at least one physical component originating from waste material.

As used herein, the terms “including,” “include,” and “included” have the same open-ended meaning as “comprising,” “comprises,” and “comprise” provided above.

As used herein, the term “indirectly derived” refers to having an applied recycled content (i) that is attributable to waste material, but (ii) that is not based on having a physical component originating from waste material.

As used herein, the term “located remotely” refers to a distance of at least 0.1, 0.5, 1, 5, 10, 50, 100, 500, or 1000 miles between two facilities, sites, or reactors.

As used herein, the term “mass balance” refers to a method of tracking recycled content based on the mass of the recycled content in various materials.

As used herein, the terms “physical recycled content” and “direct recycled content” both refer to matter that is physically traceable back to a waste material.

As used herein, the term “predominantly” means more than 50 percent by weight. For example, a predominantly propane stream, composition, feedstock, or product is a stream, composition, feedstock, or product that contains more than 50 weight percent propane.

As used herein, the term “recycled content” refers to being or comprising a composition that is directly and/or indirectly derived from recycle material. Recycled content is used generically to refer to both physical recycled content and credit-based recycled content. Recycled content is also used as an adjective to describe material having physical recycled content and/or credit-based recycled content.

As used herein, the term “recycled content credit” refers to a non-physical measure of physical recycled content that can be directly or indirectly (i.e., via a digital inventory) attributed from a first material having physical recycled content to a second material having less than 100 percent physical recycled content.

As used herein, the term “total recycled content” refers to the cumulative amount of physical recycled content and credit-based recycled content from all sources.

As used herein, the term “waste material” refers to used, scrap, and/or discarded material.

As used herein, the terms “waste plastic” and “plastic waste” refer to used, scrap, and/or discarded plastic materials.

### SPECIFIC EMBODIMENTS

Embodiment 1. A process for producing an oxo product having recycled content, the process comprising: hydroformylating a C2 to C24 olefin with syngas to provide a first oxo product, wherein the first oxo product comprises recycled content obtained directly or indirectly from waste plastic subjected to carbon reforming.

Embodiment 2. A process for producing an oxo product having recycled content, the process comprising: carbon reforming a hydrocarbon containing feed to thereby produce a first syngas; hydroformylating a C2 to C24 olefin with at least a portion of the first syngas to thereby produce a C3 to C25 aldehyde; and subjecting at least a portion of the C3 to C25 aldehyde to one or more additional reactions with an additional reactant to provide another oxo product, wherein the another oxo product comprises recycled content from one or more of the following sources: (i) waste plastic, (ii) a recycled content syngas, and (iii) a recycled content additional reactant.

Embodiment 3. A process for producing an oxo product or a derivative thereof having recycled content, the process comprising: carbon reforming a hydrocarbon containing feed to thereby produce a first syngas;
hydroformylating a C2 to C24 olefin with at least a portion of the first syngas to thereby produce a first oxo product; and subjecting at least a portion of the first oxo product to at least one additional reaction with an additional reactant to thereby produce a second oxo product, wherein the second oxo product comprises recycled content from one or more of the following sources: (i) waste plastic, (ii) a recycled content syngas, and (iii) a recycled content additional reactant.

Embodiment 4. A system or package comprising: an oxo product and an identifier associated with the oxo product, wherein the identifier is a representation that the oxo product has recycled content or is made from a source having recycled content.

Embodiment 5. Use of a recycled content syngas to produce a recycled content oxo product.

Embodiment 6. A recycled content end product comprising a recycled content oxo product and at least one other material.

Embodiment 7. The process, system, or product of any of Embodiments 1-6, further comprising reacting the oxo product with at least additional reactant to thereby produce a second recycled content oxo product (r-oxo product). Embodiment 8. The process, system, or product of any of Embodiments 1-7, wherein the additional reactant comprises a recycled content additional reactant (r-reactant). Embodiment 9. The process, system, or product of any of Embodiments 1-8, wherein the r-reactant comprises recycled content DMT (r-DMT). Embodiment 10. The process, system, or product of any of Embodiments 1-9, wherein the r-DMT comprises recycled content obtained directly or indirectly from the solvolysis of waste plastic. Embodiment 11. The process, system, or product of any of Embodiments 1-10, wherein the r-reactant comprises recycled content ethylene glycol (r-EG). Embodiment 12. The process, system, or product of any of Embodiments 1-11, wherein the r-EG comprises recycled content obtained directly or indirectly from the solvolysis of waste plastic. Embodiment 13. The process, system, or product of any of Embodiments 1-12, wherein the r-reactant comprises one or more of the following compounds having recycled content hydrogen (r-H2), recycled content formaldehyde (r-formaldehyde), recycled content acetaldehyde (r-acetaldehyde), recycled content ethylene oxide (r-ethylene oxide), recycled content acetic acid (r-acetic acid), recycled content acetic anhydride (r-acetic anhydride), recycled content acetone (r-acetone), and recycled content benzoic acid (r-benzoic acid). Embodiment 14. The process, system, or product of any of Embodiments 1-13, wherein the additional reactant is a sustainable reactant (s-reactant). Embodiment 15. The process, system, or product of any of Embodiments 1-14, wherein the s-reactant comprises one or more of cellulose, naturally-occurring acids, and naturally occurring alcohols. Embodiment 16. The process, system, or product of any of Embodiments 1-15, wherein the oxo product comprises recycled content obtained directly from waste plastic subjected to carbon reforming. Embodiment 17. The process, system, or product of any of Embodiments 1-16, wherein the oxo product comprises recycled content obtained indirectly from waste plastic subjected to carbon reforming. Embodiment 18. The process, system, or product of any of Embodiments 1-17, wherein the oxo product comprises recycled content obtained both directly and indirectly from waste plastic subjected to carbon reforming. Embodiment 19. The process, system, or product of any of Embodiments 1-18, wherein the olefin is a C2 olefin and the oxo product is a C3 aldehyde. Embodiment 20. The process, system, or product of any of Embodiments 1-19, further comprising, subjecting the C3 aldehyde to at least one additional reaction to thereby form a second oxo product. Embodiment 21. The process, system, or product of any of Embodiments 1-20, wherein the second oxo product includes one or more of the following compounds: n-propanol, n-propyl acetate, glycol ether, n-propyl propionate, cellulose acetate propionate, propionic acid, and propionic anhydride. Embodiment 22. The process, system, or product of any of Embodiments 1-21, wherein the olefin is a C3 olefin and the oxo product is a C4 aldehyde. Embodiment 23. The process, system, or product of any of Embodiments 1-22, further comprising, subjecting the C4 aldehyde to at least one additional reaction to thereby form a second oxo product. Embodiment 24. The process, system, or product of any of Embodiments 1-23, wherein the second oxo product includes at least one of 2-ethylhexanol, 2-ethylhexaldehyde, 2-ethylhexylacid, tri(ethylene glycol)bis(2-ethanolhexanoate), and 2-ethylhexanol acetate. Embodiment 25. The process, system, or product of any of Embodiments 1-24, wherein the second oxo product includes at least one of dipropylene glycol dibenzoate, bis(2-ethylhexyl) maleate, bis(2-ethylhexyl) terephthalate, dioctyl phthalate, bis(2-ethylhexyl) adipate, oxydiethylene dibenzoate, tris(2-ethylhexyl)trimellitate, and dibutylterephthalate. Embodiment 26. The process, system, or product of any of Embodiments 1-25, wherein the second oxo product includes one or more of the following compounds: methyl n-amyl ketone (MAK), n-butyric acid, normal butranitrile, butyric anhydride, and cellulose acetate butyrate. Embodiment 27. The process, system, or product of any of Embodiments 1-26, wherein the second oxo product includes one or more of the following compounds: n-butanol, n-butyl propionate, n-butyl acetate, n-butyraldehyde, ethylene glycol monobutyl ether, diethylene glycol monobutyl ether, ethylene glycol monopropyl ether, ethylene glycol dipropyl ether, ethylene glycol 2-ethylhexyl ether, glycol ether esters (ethylene glycol monobutyl ether ester, diethylene glycol monobutyl ether ester, and diethylene glycol monoethyl acetate). Embodiment 28. The process, system, or product of any of Embodiments 1-27, wherein the second oxo product includes one or more of the following compounds: methyl isoamyl ketone, neopentyl glycol, 2,2,4-trimethyl-1,3-pentanediol monoisobutyrate, 2,2,4-trimethyl-1,3-pentanediol diisobutyrate, 2,2,4-trimethyl-1,3-pentanediol, isobutyl isobutyrate, isobutyl alcohol, isobutyl acetate, 2,2,4,4-tetramethyl-1,3-cyclobutanediol, isobutyric anhydride, isobutyronitrile, and methyl isopropyl ketone. Embodiment 29. The process, system, or product of any of claims 1-28, wherein the second oxo product includes a copolyester. Embodiment 30. The process, system, or product of any of Embodiments 1-29, wherein the C4 aldehyde is at least 50, 75, 90 percent n-C4 aldehyde. Embodiment 31. The process, system, or product of any of Embodiments 1-30, wherein the C4 aldehyde is at least 50, 75, 90 percent i-C4 aldehyde. Embodiment 32. The process, system, or product of any of Embodiments 1-31, further comprising, reacting the C4 aldehyde with at least one additional reactant to thereby produce a second oxo product. Embodiment 33. The process, system, or product of any of Embodiments 1-2, further comprising, hydrogenating the C4 aldehyde to provide a C4 alcohol. Embodiment 34. The process, system, or product of any of Embodiments 1-33, wherein the hydrogen used to carry out the hydrogenation is recycled content hydrogen (r-H2). Embodiment 35. The process, system, or product of any of Embodiments 1-34, further comprising, reacting the C4 alcohol with at least one additional reactant to thereby produce a second oxo product. Embodiment 36. The process, system, or product of any of Embodiments 1-35, wherein the oxo product is a C3 to C25 aldehyde. Embodiment 37. The process, system, or product of any of Embodiments 1-36, further comprising subjecting the aldehyde to at least one additional reaction to form a second oxo product. Embodiment 38. The process, system, or product of any of Embodiments 1-37, further comprising subjecting the second oxo product to another additional reaction with at least one additional reactant to thereby produce a second oxo product.

Embodiment 39. The process, system, or product of any of Embodiments 1-38, wherein the additional reactant has recycled content or is sustainable. Embodiment 40. The process, system, or product of any of Embodiments 1-39, further comprising hydrogenating at least a portion of the C3 to C25 aldehyde to form an alcohol. Embodiment 41. The process, system, or product of any of Embodiments 1-40, further comprising subjecting the alcohol to at least one additional reaction to form a second oxo product. Embodiment 42. The process, system, or product of any of Embodiments 1-41, wherein the hydrogenating is carried out with recycled content hydrogen (r-H2). Embodiment 43. The process, system, or product of any of Embodiments 1-42, wherein the syngas is a recycled content syngas (r-syngas). Embodiment 44. The process, system, or product of any of Embodiments 1-43, further comprising carbon reforming a recycled content hydrocarbon feed (r-HC feed) to provide the r-syngas. Embodiment 45. The process, system, or product of any of Embodiments 1-44, wherein the r-HC feed includes non-recycled content hydrocarbon. Embodiment 46. The process, system, or product of any of Embodiments 1-45, wherein the non-recycled content hydrocarbon includes coal. Embodiment 47. The process, system, or product of any of Embodiments 1-46, wherein the r-HC feed includes waste plastic. Embodiment 48. The process, system, or product of any of Embodiments 1-47, wherein the r-HC feed includes recycled content pyrolysis oil (r-pyoil) formed from pyrolysis of waste plastic. Embodiment 49. The process, system, or product of any of Embodiments 1-48, wherein the carbon reforming comprises partial oxidation gasification. Embodiment 50. The process, system, or product of any of Embodiments 1-49, wherein the carbon reforming comprises catalytic reforming. Embodiment 51. The process, system, or product of any of Embodiments 1-50, wherein the carbon reforming comprises steam reforming. Embodiment 52. The process, system, or product of any of Embodiments 1-51, wherein the carbon reforming comprises plasma gasification. Embodiment 53. The process, system, or product of any of Embodiments 1-52, wherein the carbon reforming produces a recycled content hydrogen (r-H2) and further comprising hydrogenating at least a portion of the oxo product with r-H2. Embodiment 54. The process, system, or product of any of Embodiments 1-53, wherein the olefin is a recycled content olefin (r-olefin) formed by pyrolysis of waste plastic. Embodiment 55. The process, system, or product of any of Embodiments 1-54, further comprising pyrolyzing waste plastic to form recycled content pyrolysis oil (r-pyoil) and recycled content pyrolysis gas (r-pygas), wherein the r-olefin is formed from at least one of the r-pyoil and r-pygas. Embodiment 56. The process, system, or product of any of Embodiments 1-55, wherein the r-olefin is produced by cracking the r-pyoil. Embodiment 57. The process, system, or product of any of Embodiments 1-56, wherein the r-olefin is produced by separating the r-pygas. Embodiment 58. The process, system, or product of any of Embodiments 1-58, wherein the oxo product or derivative is an alcohol, a carboxylic acid, a ketone, an ester, an amide, an ether, an amine, an olefin (alkene), or a paraffin (alkane). Embodiment 59. The process, system, or product of any of Embodiments 1-58, wherein the oxo product or derivative is a C3 to C50, C3 to C35, C3 to C30 hydrocarbon compound. Embodiment 60. The process, system, or product of any of Embodiments 1-59, wherein the oxo product or derivative has a total recycled content of at least 50, 55, 60, 65, 70, 75, 80, 85, 90, or 95 percent. Embodiment 61. The process, system, or product of any of Embodiments 1-60, wherein the oxo product or derivative comprise a polymer. Embodiment 62. The process, system, or product of any of Embodiments 1-61, wherein the second oxo product comprises a solvent, a surfactant, a plasticizer, a coalescent, a dye, a fragrance, a lubricant, or a polymerizing agent.

Embodiment 63. An end product comprising an oxo product or second oxo product and at least one other material, wherein the end product is selected from the group consisting of personal care product, a fragrance, detergent, cosmetic, a solvent, a coating, a paint, a fiber, a packaging material, a bottle, a container, a sheet, construction materials, a medical product (e.g., eyewear), an electronic product (e.g., displays), a food additive (e.g., preservative), an automobile component (e.g., plastics, carpets, windshield interlayers, fluids).

Embodiment 64. The process, system, or product of any of Embodiments 1-63, wherein the end product is for medical end use, consumer end use, or industrial end use. Embodiment 65. The process, system, or product of any of Embodiments 1-64, wherein the oxo product comprises physical recycled content from one or more of the source materials. Embodiment 66. The process, system, or product of any of Embodiments 1-65, wherein the oxo product comprises credit-based recycled content from one or more of the source materials. Embodiment 67. The process, system, or product of any of Embodiments 1-66, wherein the oxo product comprises physical and credit-based recycled content from one or more of the source materials. Embodiment 68. The process, system, or product of any of Embodiments 1-67, wherein the oxo product comprises recycled content from the r-syngas. Embodiment 69. The process, system, or product of any of Embodiments 1-68, wherein the oxo product comprises physical recycled content from the r-syngas. Embodiment 70. The process, system, or product of any of Embodiments 1-69, wherein the oxo product comprises credit-based recycled content from the r-syngas. Embodiment 71. The process, system, or product of any of Embodiments 1-70, wherein the oxo product comprises recycled content from the r-H2. Embodiment 72. The process, system, or product of any of Embodiments 1-71, wherein the oxo product comprises physical recycled content from the r-H2. Embodiment 73. The process, system, or product of any of Embodiments 1-72, wherein the oxo product comprises credit-based recycled content from the r-H2. Embodiment 74. The process, system, or product of any of Embodiments 1-73, wherein the oxo product comprises recycled content from the r-reactant. Embodiment 75. The process, system, or product of any of Embodiments 1-74, wherein the oxo product comprises physical recycled content from the r-reactant. Embodiment 76. The process, system, or product of any of Embodiments 1-75, wherein the carbon reforming, hydroformylating and additional reactions are carried out in an oxo product production facility, wherein at least one of the source materials providing recycled content to the oxo product is produced in a remote source facility that is located at least 0.5, 1, 5, 10, 100, 500, 1000 or 10000 miles from the oxo product production facility. Embodiment 77. The process, system, or product of any of Embodiments 1-76, wherein the oxo product comprises credit-based recycled content from the at least one source material produced in the remote source facility. Embodiment 78. The process, system, or product of any of Embodiments 1-77, wherein the carbon reforming, hydroformylating and additional reactions are carried out in an oxo product production facility, wherein at least one of the source materials is produced in a co-located source facility located within 5, 1, 0.5, or 0.25 miles of the oxo product production facility. Embodiment 79. The process, system, or product of any of Embodiments 1-78, wherein the oxo product comprises physical recycled content from the at least on source material produced in the remote source facility. Embodiment 80. The process, system, or product of any of Embodiments 1-79, further comprising apply credit-based recycled content to the oxo product from the one or more source materials. Embodiment 81. The process, system, or product of any of Embodiments 1-80, wherein the applying includes (i) attributing recycled content from at least one of the source materials having physical recycled content to at least one target material via recycled content credits, (ii) tracing recycled content along at least one chemical pathway from the at least one target material to the oxo product, and (iii) allocating recycled content to the oxo product based at least in part on the tracing of recycled content along the chemical pathway. Embodiment 82. The process, system, or product of any of Embodiments 1-81, wherein at least one of the following criterial is met (i) the source material and the target material both comprise syngas and (ii) the source material and the target material both comprise hydrogen. Embodiment 83. The process, system, or product of any of Embodiments 1-82, wherein the attributing includes (i) booking recycled content credits attributable to the at least one source material into a digital inventory and (ii) assigning recycled content credits from the digital inventory to the target material. Embodiment 84. The process, system, or product of any of Embodiments 1-83, wherein the tracing includes determining one or more conversion factors for one or more chemical reactions along the chemical pathway, wherein the attributing includes assigning credit-based recycled content from a digital inventory to the target material, wherein the conversion factors determine how much of the credit-based recycled content applied to the target material is allocated to the oxo product.

CLAIMS NOT LIMITED TO DISCLOSED EMBODIMENTS

The preferred forms of the invention described above are to be used as illustration only and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

1. A process for producing an oxo product or a derivative thereof having recycled content, the process comprising:
(a) carbon reforming a hydrocarbon containing feed to thereby produce a first syngas;
(b) hydroformylating a C2 to C24 olefin with at least a portion of the first syngas to thereby produce a first oxo product; and
(c) subjecting at least a portion of the first oxo product to at least one additional reaction with an additional reactant to thereby produce a second oxo product,
wherein the second oxo product comprises recycled content from one or more of the following sources: (i) waste plastic, (ii) a recycled content syngas (r-syngas), and (iii) a recycled content additional reactant.

2. A system or package comprising: an oxo product and an identifier associated with the oxo product, wherein the identifier is a representation that the oxo product has recycled content or is made from a source having recycled content.

3. The process claim 1, wherein the additional reactant comprises a recycled content additional reactant (r-reactant).

4. The process of claim 1, wherein the r-reactant comprises recycled content dimethyl terephthalate (r-DMT), recycled content ethylene glycol (r-EG).

5. The process claim 1, wherein the additional reactant is a sustainable reactant (s-reactant).

6. The process of claim 1, wherein the oxo product comprises recycled content obtained directly and/or indirectly from waste plastic subjected to carbon reforming.

7. The process of claim 1, wherein the olefin is a C2 olefin and the first oxo product is a C3 aldehyde.

8. The process of claim 1, wherein the olefin is a C3 olefin and the first oxo product is a C4 aldehyde.

9. The process of claim 1; system, wherein the second oxo product includes one or more of the following compounds: n-propanol, n-propyl acetate, glycol ether, n-propyl propionate, cellulose acetate propionate, propionic acid, and propionic anhydride, 2-ethylhexanol, 2-ethylhexaldehyde, 2-ethylhexylacid, tri(ethylene glycol)bis(2-ethanolhexanoate), 2-ethylhexanol acetate, dipropylene glycol dibenzoate, bis(2-ethylhexyl) maleate, bis(2-ethylhexyl) terephthalate, dioctyl phthalate, bis(2-ethylhexyl) adipate, oxydiethylene dibenzoate, tris(2-ethylhexyl)trimellitate, dibutylterephthalate, methyl n-amyl ketone (MAK), n-butyric acid, normal butranitrile, butyric anhydride, cellulose acetate butyrate, n-butanol, n-butyl propionate, n-butyl acetate, n-butyraldehyde, ethylene glycol monobutyl ether, diethylene glycol monobutyl ether, ethylene glycol monopropyl ether, ethylene glycol dipropyl ether, ethylene glycol 2-ethylhexyl ether, glycol ether esters (ethylene glycol monobutyl ether ester, diethylene glycol monobutyl ether ester, and diethylene glycol monoethyl acetate), ketone, methyl isoamyl neopentyl glycol, 2,2,4-trimethyl-1,3-pentanediol monoisobutyrate, 2,2,4-trimethyl-1,3-pentanediol diisobutyrate, 2,2,4-trimethyl-1,3-pentanediol, isobutyl isobutyrate, isobutyl alcohol, isobutyl acetate, 2,2,4,4-tetramethyl-1,3-cyclobutanediol, isobutyric anhydride, isobutyronitrile, and methyl isopropyl ketone, and copolyester.

10. The process claim 1, wherein the additional reactant has recycled content or is sustainable.

11. The process of claim 1, further comprising carbon reforming a recycled content hydrocarbon feed (r-HC feed) to provide the r-syngas.

12. The process of claim 1, wherein the carbon reforming comprises partial oxidation gasification, or catalytic reforming, or steam reforming, or plasma gasification.

13. The process of claim 1, wherein the carbon reforming produces a recycled content hydrogen (r-H2) and further comprising hydrogenating at least a portion of the oxo product with r-H2.

14. The process of claim 1, wherein the olefin is a recycled content olefin (r-olefin) formed by pyrolysis of waste plastic, methanol, or cracking of hydrocarbons.

15. The process, wherein the oxo product or derivative is an alcohol, a carboxylic acid, a ketone, an ester, an amide, an ether, an amine, an olefin (alkene), or a paraffin (alkane).

16. The process of claim 1, wherein the oxo product comprises physical recycled content from one or more of the source materials and/or credit-based recycled content from one or more of the source materials.

17. The process of claim 1, wherein the oxo product comprises recycled content from the r-syngas, or physical recycled content from the r-syngas, or credit-based recycled content from the r-syngas or recycled content from the r-H2, or physical recycled content from the r-H2 or credit-based recycled content from the r-H2, or recycled content from the r-reactant or physical recycled content from the r-reactant, or a combination.

18. The process of claim 1, wherein the carbon reforming, hydroformylating and additional reactions are carried out in an oxo product production facility, wherein at least one of the source materials providing recycled content to the oxo product is produced in a remote source facility that is located at least 0.5, 1, 5, 10, 100, 500, 1000 or 10000 miles from the oxo product production facility or a co-located source facility located within 5, 1, 0.5, or 0.25 miles of the oxo product production facility.

19. The process of claim 1, wherein the oxo product comprises credit-based recycled content from the at least one source material produced in the remote source facility.

20. The process of claim 1, further comprising apply credit-based recycled content to the oxo product from the one or more source materials.
